(19) United States
(12) Patent Application Publication
(10) Pub. No.: US 2006/0040290 A1
(43) Pub. Date: Feb. 23, 2006

(54) Methods, compositions, and kits comprising PNA for RNA interference

(75) Inventor: Gerald Zon, San Carlos, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/166,031

(22) Filed: Jun. 23, 2005

Related U.S. Application Data
(60) Provisional application No. 60/582,608, filed on Jun. 23, 2004.

(51) Int. Cl. A61K 48/00; C12Q 1/68

(52) U.S. Cl. 435/6; 514/12; 514/44

(57) ABSTRACT

The present teachings relate to methods, compositions, and kits for reducing the amount of a target polynucleotide sequence. In some embodiments, block oligomers comprising peptide nucleic acids (PNAs) are hybridized to an anti-sense strand to form an anti-sense complex comprising regions with desirable thermodynamic characteristics. In some embodiments, the expression of a target messenger RNA is reduced in RNA interference (RNAi) experiments using anti-sense compositions comprising PNAs.

### CLAIM TO PRIORITY

This application claims priority benefit under 35 U.S.C. § 119(e) from U.S. Provisional Patent Application No. 60/582,608, filed Jun. 23, 2004, which is incorporated herein by reference.

### FIELD

The present teachings generally relate to methods, kits, and compositions for reducing the expression of target polynucleotide sequences. More specifically, the teachings relate to methods, kits, and compositions comprising peptide nucleic acids with desirable thermodynamic characteristics for performing RNA interference studies.

### BACKGROUND

RNA interference (RNAi) refers to an intracellular process by which ˜21 nucleotide ribooligonucleotides called small interfering RNAs (siRNAs) inhibit gene expression by sequence-specific cleavage of mRNA. RNAi has attracted considerable recent interest in various fields of basic science, in validation of gene targets for small molecule drug discovery, and as a therapeutic modality per se (for review see Milhavet et al., Pharmacological Reviews, 2003, 55: 629-648, Brummelkamp et al., Nature Review Cancer, 2003: 3: 781-789, Zhang et al., Curr Pharm Biotechnol. 2004 February; 5(1):1-7, Kittler et al., Semin Cancer Biol. 2003 August; 13(4):259-65.). However, the facile widespread application of RNAi is limited by a number of problems, including a background interferon response that can be toxic to cells, inhibition of the expression of genes other than those being targeted (so called “off-target effects”), and cost-prohibitive economics of large-scale RNA synthesis. There is a great interest in rapid, effective, and inexpensive approaches to manipulating gene expression in RNAi applications.

Procedures known in the art for selecting the appropriate region of RNA for RNAi involve the use of algorithms to select for those sequences with minimal secondary and tertiary structure, those targets with minimal sequence redundancy with other regions of the genome, those target regions with desirable thermodynamic characteristics, and other parameters desirable for the context at hand. For example, commercially available algorithms useful for selecting appropriate target regions of target messenger RNA can be obtained from Ambion, Qiagen, Dharmacon-Fisher Scientific (siDESIGN CENTER), Sequitur, Alnylam, Cenix, and others, also see for example Gonczy et al., Nature 408:331, 2000, Harborth et al., Antisense Nucleic Acid Drug Dev. 2003 April; 13(2):83-105. Nonetheless, it is recognized that better sequence selection procedures are needed in order to more effectively choose target regions for the selective knock-down of the expression of target genes with RNAi.

One illustrative gene that RNAi interference could effectively reduce the expression of a target messenger RNA is the human multidrug resistance gene (MDR1). MDR1 spans >200 kb and encodes the ATP-dependent membrane efflux transporter, P-glycoprotein (P-gp), and plays a key role in both anticancer and antiviral therapy because of its modulation of intracellular drug concentration (Woodahl and Ho 2004, Current Drug Metabolism, 5:11-19; Kim, 2003, Top. HIV Med. 11:136-139). Multidrug resistance, which is a major problem in cancer chemotherapy, is believed to be the phenotype of the cellular overproduction of P-gp (Nieth et al., 2003, REBS Lett. 545:144-150). A disruption of P-gp-mediated drug efflux results in a re-sensitization of tumor cells to treatment with anti-neoplastic agents, and thus may allow a successful drug treatment of the multidrug-resistance cancer cells (Kuss et al., 2002, International Journal Cancer, 98:128-133). A phosphorothioate (PS) antisense oligonucleotide (AS-ODN) targeting MDR1 has been shown to produce chemosensitzation toward etoposide-resistant cancer cells but required at least 1 uM concentration, which is not a realistically sustainable level of PS AS-ODN concentration in vivo (Webb and Zon, 1999, Current Opinion Molecular Therapies, 1:458-463). RNAi activity of siRNAs targeted to MDR1 have also been reported (Wu et al., 2003, Cancer Research, 63:1515-1519, Nieth et al., 2003); however in vivo use of siRNA constructs may be compromised by off-target effects and/or interferon-like side-effects. Further, significant progress has been made in the discovery of MDR1 polymorphisms and the assessment of allelic frequencies (Woodahl and Ho, 2004 Current Drug Metabolism, 5:11-19). For example, a single nucleotide polymorphism (SNP) in MDR1 such as the G267T/A (Ala893Ser/Thr) in exon 26 has been studied with regard to alteration of the disposition of pharmacokineteics of drugs (Siegmund et al., 2002, Clin Pharm. Therap., 72(5):572-583, Verstuyft et al, 2003, J. American Soc. of Nephrology, 14(7):1889-1896).

Peptide Nucleic Acids (PNAs) are a non-naturally occurring polyamide (pseudopeptide) that can hybridize to nucleic acids with sequence specificity (see for example U.S. Pat. No. 5,539,082 and Egholm et al., Nature 365: 566-568 (1993)). PNAs are promising candidates for the sequence-specific regulation of polynucleotide target sequences and for the preparation of gene targeted drugs (See European Patent applications EP 92/01219 and 92/01220).

### SUMMARY

In some embodiments, the present teachings provide a method of reducing the amount of a target polynucleotide sequence comprising providing an anti-sense strand, wherein the anti-sense strand is complementary to a target region, and, at least two block oligomers, wherein the at least two block oligomers further comprise PNA, wherein the block oligomer can hybridize to the anti-sense strand to form an anti-sense complex. The antisense complex is delivered to a sample comprising the target polynucleotide sequence, and the amount of the target polynucleotide sequence is reduced.

In some embodiments, the present teachings provide a method of reducing the amount of at least one target polynucleotide sequence comprising providing an anti-sense strand, wherein the anti-sense strand is complementary to a target region, and, a combination oligomer, wherein the combination oligomer further comprises a PNA nucleobase sequence, a first RNA nucleobase sequence, and a second RNA nucleobase sequence, wherein the combination oligomer hybridizes to the anti-sense strand to form an anti-sense complex. The anti-sense complex is delivered to a sample comprising the target polynucleotide sequence, and the amount of the target polynucleotide sequence is reduced.

In some embodiments, the present teachings provide a composition of matter comprising an anti-sense strand complementary to a target region, and, a first block oligomer, a second block oligomer, and a third block oligomer, wherein the first block oligomer and the third block oligomer each comprise an RNA nucleobase sequence, and the second block oligomer comprises a PNA nucleobase sequence, wherein the first block oligomer, the second block oligomer, and the third block oligomer are hybridized to the anti-sense strand.

In some embodiments, the present teachings provide a kit for reducing the amount of a target polynucleotide sequence comprising an anti-sense complex, a transfection agent, and competent cells.

### BRIEF DESCRIPTION OF THE DRAWINGS

FIG. 1 depicts certain method embodiments of the present teachings.

FIG. 2 depicts certain method embodiments of the present teachings.

FIG. 3 depicts certain method embodiments of the present teachings.

FIG. 4 depicts certain method embodiments of the present teachings.

FIG. 5 depicts certain method embodiments of the present teachings.

FIG. 6 depicts certain method embodiments of the present teachings.

FIG. 7 depicts certain method embodiments of the present teachings.

FIG. 8 depicts certain method embodiments of the present teachings.

FIG. 9 depicts certain method embodiments of the present teachings.

FIG. 10 depicts certain composition embodiments of the present teachings, wherein “P” indicates PNA, “R” indicates RNA, and the strands of the anti-sense complex have the strand polarity and subunit position numbering as indicated.

Table 1 illustrates certain thermodynamic profiles according to some embodiments of the present teachings.

### DESCRIPTION OF EXEMPLARY EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall prevail.

It is to be understood that both the foregoing general description and the following detailed description are exemplary and explanatory only and are not restrictive of the present teachings. In this application, the use of the singular includes the plural unless specifically stated otherwise. Also, the use of “or” means “and/or” unless the context dictates otherwise. Similarly, “comprise”, “comprises”, “comprising”, “include”, “includes”, and “including” are not intended to be limiting.

1. Definitions

As used herein, the “probes,” “primers,” “targets,” and “oligomers” of the present teachings can be comprised of at least one of ribonucleotides, deoxynucleotides, modified ribonucleotides, modified deoxyribonucleotides, modified phosphate-sugar-backbone oligonucleotides, nucleotide analogs, and combinations thereof.

The term “nucleotide”, as used herein, generically encompasses the following terms, which are defined below: nucleotide base, nucleoside, nucleotide analog, extendable nucleotide, and universal nucleotide.

The term “nucleotide base”, as used herein, refers to a substituted or unsubstituted parent aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, purines such as 2-aminopurine, 2,6-diaminopurine, adenine (A), ethenoadenine, N6-Δ2-isopentenyladenine (61A), N6-Δ2-isopentenyl-2-methylthioadenine (2 ms61A), N6-methyladenine, guanine (G), isoguanine, N2dimethylguanine (dmG), 7-methylguanine (7mG), 2-thiopyrimidine, 6-thioguanine (6sG) hypoxanthine and O6-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O4-methylthymine, uracil (U), 4-thiouracil (4sU) and 5,6-dihydrouracil (dihydrouracil; D); indoles such as nitroindole and 4-methylindole; pyrroles such as nitropyrrole; and nebularine; etc. In certain embodiments, nucleotide bases are universal nucleotide bases. Additional exemplary nucleotide bases can be found, e.g., in Fasman, 1989, Practical Handbook of Biochemistry and Molecular Biology, pp. 385-394, CRC Press, Boca Raton, Fla., and the references cited therein. Further examples of universal bases can be found for example in Loakes, N.A.R. 2001, vol 29:2437-2447 and Seela N.A.R. 2000, vol 28:3224-3232.

The term “nucleoside”, as used herein, refers to a compound having a nucleotide base covalently linked to the C-1′ carbon of a pentose sugar. In certain embodiments, the linkage is via a heteroaromatic ring nitrogen. Typical pentose sugars include, but are not limited to, those pentoses in which one or more of the carbon atoms are each independently substituted with one or more of the same or different —R, —OR, —NRR or halogen groups, where each R is independently hydrogen, (C1-C6) alkyl or (C5-C14) aryl. The pentose sugar may be saturated or unsaturated. Exemplary pentose sugars and analogs thereof include, but are not limited to, ribose, 2′-deoxyribose, 2′-(C₁-C₆)alkoxyribose, 2′-(C5-C14)aryloxyribose, 2′,3′-dideoxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C₁-C₆)alkylribose, 2′-deoxy-3′-(C1-C6)alkoxyribose and 2′-deoxy-3′-(C5-C₁₋₄)aryloxyribose. Also see e.g. 2′-O-methyl, 4′-α-anomeric nucleotides, 1′-α-anomeric nucleotides (Asseline (1991) Nucl. Acids Res. 19:4067-74), 2′-4′- and 3′-4′-linked and other “locked” or “LNA”, bicyclic sugar modifications (WO 98/22489; WO 98/39352; WO 99/14226). “LNA” or “locked nucleic acid” is a DNA analogue that is conformationally locked such that the ribose ring is constrained by a methylene linkage between the 2′-oxygen and the 3′- or 4′-carbon. The conformation restriction imposed by the linkage often increases binding affinity for complementary sequences and increases the thermal stability of such duplexes.

Exemplary LNA sugar analogs within a polynucleotide include the structures: 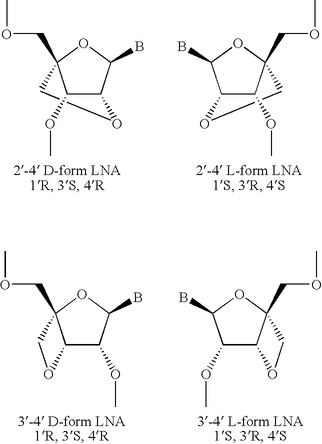
where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleobase is purine, e.g. A or G, the ribose sugar is attached to the N⁹-position of the nucleobase. When the nucleobase is pyrimidine, e.g. C, T or U, the pentose sugar is attached to the N¹-position of the nucleobase (Kornberg and Baker, (1992) DNA Replication, 2^{nd} Ed., Freeman, San Francisco, Calif.).

One or more of the pentose carbons of a nucleoside may be substituted with a phosphate ester having the formula: 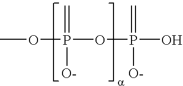
where α is an integer from 0 to 4. In certain embodiments, α is 2 and the phosphate ester is attached to the 3′- or 5′-carbon of the pentose. In certain embodiments, the nucleosides are those in which the nucleotide base is a purine, a 7-deazapurine, a pyrimidine, a universal nucleotide base, a specific nucleotide base, or an analog thereof.

The term “nucleotide analog”, as used herein, refers to embodiments in which the pentose sugar and/or the nucleotide base and/or one or more of the phosphate esters of a nucleoside may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions. Other nucleic acid analogs and bases include for example intercalating nucleic acids (INAs, as described in Christensen and Pedersen, 2002), and AEGIS bases (Eragen, U.S. Pat. No. 5,432,272).

The term “universal nucleotide base” or “universal base”, as used herein, refers to an aromatic ring moiety, which may or may not contain nitrogen atoms. In certain embodiments, a universal base may be covalently attached to the C-1′ carbon of a pentose sugar to make a universal nucleotide. In certain embodiments, a universal nucleotide base does not hydrogen bond specifically with another nucleotide base. In certain embodiments, a nucleotide base may interact with adjacent nucleotide bases on the same nucleic acid strand by hydrophobic stacking. Some examples of universal nucleotides include deoxy-7-azaindole triphosphate (d7AITP), deoxyisocarbostyril tri phosphate (dlCSTP), deoxypropynylisocarbostyril tri phosphate (dPlCSTP), deoxymethyl-7-azaindole triphosphate (dM7AlTP), deoxylmPy triphosphate (dlmPyTP), deoxyPP triphosphate (dPPTP), or deoxypropynyl-7-azaindole triphosphate (dP7AlTP). Further examples of such universal bases can be found, inter alia, in Published P.C.T. Application US02/33619, and U.S. Pat. No. 6,433,134. Universal bases can also be PNA-bases, as can be found for example in U.S. Pat. No. 6,433,134.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides have associated counter ions, such as H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. Polynucleotides may be comprised of internucleotide, nucleobase and/or sugar analogs. Polynucleotides typically range in size from a few monomeric units, e.g. 3-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine, “G” denotes deoxyguanosine, and “T” denotes thymidine, unless otherwise noted. Some examples of oligonucleotides and polynucleotides in the present teachings include probes, primers, targets, and oligomers.

As used herein, “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids. Non-limiting examples of suitable nucleobases include: adenine, cytosine, guanine, thymine, uracil, 5-propynyl-uracil, 2-thio-5-propynyl-uracil, 5-methlylcytosine, pseudoisocytosine, 2-thiouracil and 2thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobase include those nucleobases illustrated in FIGS. 2(A) and 2(B) of Buchardt et al. (WO92/20702 or WO92/20703).

As used herein, “nucleobase sequence” means any segment, or aggregate of two or more segments (e.g. the aggregate nucleobase sequence of two or more oligomer blocks), of a polymer that comprises nucleobase-containing subunits. Non-limiting examples of suitable polymers or polymers segments include oligodeoxynucleotides (e.g. DNA), oligoribonucleotides (e.g. RNA), peptide nucleic acids (PNA), PNA chimeras, PNA combination oligomers, nucleic acid analogs and/or nucleic acid mimics. When prefaced, for example a “PNA nucleobase sequence,” the nucleobase sequence comprises exclusively that kind of nucleobase (in this case, PNA).

As used herein, “polynucleobase strand” means a complete single polymer strand comprising nucleobase subunits. For example, a single nucleic acid strand of a double stranded nucleic acid is a polynucleobase strand.

As used herein, “nucleic acid” is a nucleobase sequence-containing polymer, or polymer segment, having a backbone formed from nucleotides, or analogs thereof. Preferred nucleic acids are DNA and RNA.

As used herein, “peptide nucleic acid” or “PNA” means any oligomer or polymer segment comprising at least one residue referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, and 6,107,470. The term PNA shall also apply to any oligomer or polymer segment comprising at least one subunits of those nucleic acid mimics described in among other places the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et J. Chem. Soc. Perkin Trans. 11: 547-554 (1997); Lowe et al., J. Chem. Soc. Perkin Trans. 11:555-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMS) of Shah et al. as disclosed in WO96/04000.

In some embodiments, a PNA is an oligomer or polymer segment comprising two or more covalently linked subunits of the formula found in paragraph 76 of U.S. Patent Application Publication No. 2003/0077608A1, wherein, each J is the same or different and is selected from the group consisting of H, R¹, OR¹, SR¹, NHR¹, NR¹₂, F, Cl, Br and I. Each K is the same or different and is selected from the group consisting of O, S, NH and NR¹. Each R¹ is the same or different and is an alkyl group having one to five carbon atoms that may optionally contain a heteroatom or a substituted or unsubstituted aryl group. Each A can be selected from the group comprising a single bond, a group of the formula: —(CJ₂)ₛ— and a group of the formula: —(CJ₂)ₛC(O)—, wherein, J is defined above and each s is a whole number from one to five. Each t is 1 or 2 and each u is 1 or 2. Each L is the same or different and is independently selected from: adenine, cytosine, guanine, thymine, uracil, 5-propynyl-uracil, 2-thio-5-propynyluracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine), other naturally occurring nucleobase analogs or other non-naturally occurring nucleobases.

In some embodiments, a PNA subunit comprises a naturally occurring or non-naturally occurring nucleobase attached to the N-a-glycine nitrogen of the N-[2(aminoethyl)]glycine backbone through a methylene carbonyl linkage; this currently being the most commonly used form of a peptide nucleic acid subunit.

As used herein, “target polynucleotide sequence” is a nucleobase sequence of a polynucleobase strand sought to be reduced. It is to be understood that the nature of the target sequence is not a limitation of this invention. The polynucleobase strand comprising the target sequence may be provided from any source. For example, the target sequence may exist as part of a nucleic acid (e.g. DNA or RNA), PNA, nucleic acid analog or other nucleic acid mimic. The sample containing the target sequence may be from any source, and is not a limitation of the present teachings. Generally however, the target polynucleotide sequence is a messenger RNA molecule.

As used herein, the term “anti-sense RNA strand” and “anti-sense strand” are used interchangeabley and mean a nucleobase strand complementary to a target polynucleotide sequence. In some embodiments, the target polynucleotide sequence is a sense mRNA strand. In some embodiments, the anti-sense RNA strand will be comprised of RNA, though it will be appreciated that a variety of modifications can be employed without straying from the scope of the present teachings. At least one block oligomer, at least one combination oligomer, or combinations thereof, can be hybridized to the anti-sense RNA strand according to the present teachings, thereby forming an anti-sense complex.

As used herein, the term “target region” refers to a portion of a target polynucleotide sequence that is complementary with the anti-sense RNA strand.

As used herein, the term “block oligomer” means a polymer segment comprising any of at least one PNA nucleobase, at least one RNA nucleobase, at least one DNA nucleobase, at least one combination oligomer, and combinations thereof, which can hybridize to the anti-sense RNA strand to form an anti-sense complex. Generally, block oligomers are short, typically tetramers through octamers, to facilitate large scale manufacturing of a finite set, though it will be appreciated that this is not a limitation of the present teachings. In some embodiments, a block oligomer can be designed to ligate to at least one other appropriately modified block oligomer, and/or at least one appropriately modified combination oligomer, thereby forming a combination oligomer. In some embodiments, any of the at least one PNA nucleobase, at least one RNA nucleobase, at least one DNA nucleobase, at least one combination oligomer, and combinations thereof can further comprise nucleobase analogs. A block oligomer can comprise two or more PNA subunits and one or more nucleic acid subunits (e.g. DNA or RNA) nucleobase or analogs thereof that are selected from different classes of subunits and that are linked by a covalent bond. For example, a PNA/DNA or PNA/RNA block oligomer would comprise at least one PNA subunit covalently linked, via a chemical bond, to at least one 2′-deoxyribonucleic acid subunit or at least one ribonucleic acid subunit, respectively (for exemplary methods and compositions related to PNA/DNA and PNA/RNA chimera preparation see for example PCT International Publication No. WO96/40709). In some embodiments, a block oligomer can comprise a collection of different types of nucleobase subunits (e.g. PNA, DNA, RNA) and can be formed via chemical means of stepwise monomer assembly. As used herein, when “block oligomer” is prefaced with a particular nucleobase type, it will be understood that generally other nucleobases are not present. For example, a “PNA block oligomer” comprises PNA, and generally not DNA, or RNA, or other nucleobases.

As used herein a “combination oligomer” is an oligomer comprising two or more block oligomers. In some embodiments, a combination oligomer comprising PNA can be formed via the ligation of at least one PNA block oligomer, at least DNA block oligomer, at least one RNA block oligomer, at least one chimeric block oligomer, and combinations thereof. A “DNA block oligomer” refers to a block oligomer comprising only DNA nucleobases, and analogously for PNA block oligomers, etc, whereas a “chimeric block oligomer” comprises at least two different nucleobases, for example, DNA and PNA. For a further description of combination oligomers, see for example published U.S. Patent Application Publication No. 2003/0077608. In some embodiments, a combination oligomer can be formed from the ligation of a combination oligomer with a block oligomer. In some embodiments, a combination oligomer can be formed from the ligation of a combination oligomer with another combination oligomer.

As used herein the term “oligomer” refers inclusively to both combination oligomers and block oligomers.

As used herein, the term “anti-sense complex” refers to an anti-sense strand hybridized with at least one block oligomer, at least one combination oligomer, or combinations thereof.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “sample” refers to a mixture from which the target polynucleotide sequence is derived, such sources including, but are not limited to, viruses, prokaryotes, protists, eukaryotes, plants, fungi, and animals. The samples from these sources can include, but are not limited to, whole blood, a tissue biopsy, bone marrow, amniotic fluid, hair, skin, and cultured cells.

As used herein, the term “adjacent” refers to a general proximity in the relative position of two oligomers hybridized on a template. Such adjacently hybridized oligomers may or may not be contiguous with one another. Typically, if a gap of greater than a few nucleotides exists, the oligomers would not be considered adjacent.

As used herein, the term “gap” means a space, that is at least one nucleobase in length, typically between the terminal nucleobases of at least two oligomers adjacently hybridized on a template.

As used herein, the term “contiguous” refers to the absence of a gap between the terminal nucleobases of at least two adjacently hybridized oligomers, such that the at least two oligomers are abutting one another and are potentially suitable for ligation.

As used herein, the term “complementary” refers to the ability of at least two DNA molecules, RNA molecules, nucleic acid analogs, PNAs, and combinations thereof, to hybridize together in a Watson-Crick like manner. Thus, by “complementary” it is meant that the anti-sense strand is sufficiently complementary to the target sequence to hybridize under the selected reaction conditions, though complete complementarity is not necessarily required (e.g.—there can be mismatches), though typically the anti-sense strand is completely complementary to the target sequence (e.g.—there are no mismatches). Also, “complementary” herein is meant that the oligomers are sufficiently complementary to the anti-sense RNA strand to hybridize under the selected reaction conditions, though complete complementarity is not necessarily required (e.g. there can be mismatches), though typically the oligomers are completely complementary to the anti-sense RNA strand (e.g.—there are no mismatches).

As used herein, the term “enhanced flexibility” refers to the thermodynamic characteristics of an anti-sense RNA molecule as measured in kilocalories per mole. Enhanced flexibility in the context of the present teachings generally refers to regions about 1 to about 5 of the anti-sense RNA molecule, as measured from its 5′ end, though it will be appreciated that straying somewhat from these positions are within the scope of the present teachings. In general, enhanced flexibility will comprise free energy values of about −6.5 to about −8.0 kilocalories per mole.

As used herein, the term “low internal stability” refers to the thermodynamic characteristics of a molecule as measured in kilocalories per mole. Low internal stability in the context of the present teachings generally refers to regions about 9 to about 14 of the anti-sense RNA molecule, as measured from its 5′end, though it will be appreciated that straying somewhat from these positions are within the scope of the present teachings. In general, enhanced flexibility will comprise free energy values of about −0.0 to about −9.0 kilocalories per mole.

As used herein, the term “high internal stability” refers to the thermodynamic characteristics of a molecule as measured in kilocalories per mole. High internal stability in the context of the present teachings generally refers to regions about 15 to about 19 of the anti-sense RNA molecule, as measured from its 5′ end, though it will be appreciated that straying somewhat from these positions are within the scope of the present teachings. In general, enhanced flexibility will comprise free energy values of about −8.5 to about −9.5 kilocalories per mole.

2. Techniques

PNA Assembly

Methods for the chemical assembly of PNAs are well known (See: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053 6,107,470, PerSeptive Biosystems Product Literature, and Nielsen et al., Peptide Nucleic Acids; Protocols and Applications, Horizon Scientific Press, Norfolk England (1999).

Chemicals and instrumentation for the support bound automated chemical assembly of peptide nucleic acids are now commercially available (see for example the Expedite™ FMOC PNA monomers and Reagents from Applied Biosystems). Both labeled and unlabeled PNA oligomers are likewise available from commercial vendors of custom PNA oligomers. Chemical assembly of a PNA is analogous to solid phase peptide synthesis, wherein at each cycle of assembly the oligomer possesses a reactive alkyl amino terminus that is condensed with the next synthon to be added to the growing polymer.

PNA may be synthesized at any scale, from submicromole to millimole, or more. Most conveniently, PNA is synthesized at the 2 μmole scale, using Fmoc/Bhoc, tBoc/Z, or MMT protecting group monomers on an Expedite Synthesizer (Applied Biosystems) on XAL or PAL support; or on the Model 433A Synthesizer (Applied Biosystems) with MBHA support; or on other automated synthesizers. Because standard peptide chemistry is utilized, natural and non-natural amino acids can be routinely incorporated into a PNA oligomer. Because a PNA is a polyamide, it has a C-terminus (carboxyl terminus) and an N-terminus (amino terminus). For the purposes of the design of a PNA sequence suitable for antiparallel binding to a target sequence (the preferred orientation), the N-terminus of the PNA sequence is the equivalent of the 5′-hydroxyl terminus of an equivalent DNA or RNA oligonucleotide.

Methods for synthesizing PNA-DNA and PNA-RNA chimeras are known, and can be found for example in U.S. Pat. No. 6,063,569, van der Laan et al., 1997, Tetrahedron Lettes, vol 38, no. 13, pp. 2249-2252, and Greiner et al., Helvetica Chimica Acta, 2002, Vol. 85, pp. 2619-2626, and Published P.C.T. Application WO96/40709.

Methods of making PNA libraries comprising synthesis of block oligomers are known (see for example U.S. Pat. No. 6,297,016), as are methods of ligating block oligomers together to form combination oligomers (see for example U.S. Patent Application Publication No. 2003/0077608).

Ligation

As used herein “ligation” refers to an enzymatic-based, or chemical-based, approach of connecting at least two oligomers together to form a combination oligomer, as appropriate in the context of the given embodiment of the present teachings. Non-limiting examples of numerous ligation chemistries suitable for forming combination oligomers are described in U.S. Patent Application Publication. No. 2003/0077608, FIGS. 28B and 29B-32). In some embodiments, the block oligomers can be adjacently hybridized to a template (e.g. anti-sense RNA strand), the opposing ends of the annealed block oligomers being suitable for ligation. In some embodiments, ligation can also comprise at least one preceeding gap-filling procedure, wherein the ends of the two probes are not adjacently hybridized initially but the 3′-end of the upstream block oligomer is extended by one or more nucleotide until it is adjacent to the 5′-end of the downstream oligomer, typically by a polymerase (see, e.g., U.S. Pat. No. 5,427,930 and U.S. Pat. No. 6,004,826). The internucleotide linkage can include, but is not limited to, phosphodiester bond formation. Such bond formation can include, without limitation, those created enzymatically by at least one DNA ligase or at least one RNA ligase, for example but not limited to, T4 DNA ligase, T4 RNA ligase, Thermus thermophilus (Tth) ligase, Thermus aquaticus (Taq) DNA ligase, Thermus scotoductus (Tsc) ligase, TS2126 (a thermophilic phage that infects Tsc) RNA ligase, Archaeoglobus flugidus (Afu) ligase, Pyrococcus furiosus (Pfu) ligase, or the like, including but not limited to reversibly inactivated ligases (see, e.g., U.S. Pat. No. 5,773,258), and enzymatically active mutants and variants thereof. Other internucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages. Chemical ligation can, under appropriate conditions, occur spontaneously such as by autoligation. Alternatively, “activating” or reducing agents can be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation.

Delivery of Anti-Sense Complexes into Cells

It will be appreciated that a number of known methods for introducing the compositions of the present teachings into cells can be employed in order to accomplish the methods of the present teachings. A variety of exemplary transfection procedures for introducing compositions are known including: liposome-mediated approaches described for example in U.S. 20030134423A1, and Braasch et al., Methods, 2001 23:97-107, pulsed radio frequency described for example in U.S. Pat. No. 4,970,154, composite polymers and metals-based approaches as described for example in U.S. Pat. No. 6,47,994, nucleic acid-cationic polymer-lipid mixture-based approaches as described for example in WO02100435A1, hydrogel microparticle-based approaches as described for example in WO0015263A1, and single cell based electroporation as described for example in Olofsson et al., Current Opinion in Biotechnology, 2003, 14:1:29-34 The present teachings can also be applied in the context of reverse transfection, see for example U.S. Pat. No. 6,544,790, wherein lawns of cells can be transfected with compositions of the present teachings to achieve the methods of the present teachings. In general, it will be appreciated that the nature of the delivery of the anti-sense complex is not a limitation of the present teachings.

Therapies

It will further be appreciated that the PNA-based compositions, methods, and kits of the present teachings can be used in a therapeutic setting, wherein the compositions preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient. The term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the disclosed compositions of the present teachings, its use in the therapeutic formulation is contemplated. Supplementary active ingredients can also be incorporated into the pharmaceutical formulations. It will be understood by those skilled in the art that any mode of administration, vehicle or carrier conventionally employed and which is inert with respect to the active agent can be utilized for preparing and administering disclosed compositions of the present teachings. Illustrative of such methods, vehicles and carriers are those described, for example, in Remington's Pharmaceutical Sciences, 4th ed. (1970). Those skilled in the art, having been exposed to the principles of the present teachings, will experience no difficulty in determining suitable and appropriate vehicles, excipients and carriers or in compounding the active ingredients therewith to form the disclosed compositions of the present teachings.

Universal Bases

In some embodiments of the present teachings, the RNA strand of the anti-sense complex can further comprise a universal base at a position that potentially comprises a single nucleotide polymorphism, thereby allowing for example a single anti-sense complex to hybridize to and reduce the amount of a target polynucleotide sequence (e.g. messenger RNA) comprising alternative alleles. Examples of such universal bases can be found, inter alia, in U.S. Patent Application Publication No. 10/290,672, and U.S. Pat. No. 6,433,134.

In some embodiments of the present teachings, oligomers comprise at least one universal PNA base, thereby for example providing for greater economies of scale by facilitating redundant use of a limited number of variable and universal PNA nucleobases. Some examples of universal PNA nucleobases can be found in U.S. Pat. No. 6,433,134.

Aspects of the present teachings may be further understood in light of the following exemplary embodiments, figures, and examples, which should not be construed as limiting the scope of the teachings in any way.

### 3. Exemplary Embodiments

Generation of the Anti-Sense Complex

In some embodiments, the anti-sense RNA strand can serve as a ligation substrate, such that ligation and hybridization of oligomers occur on the same anti-sense RNA strand. In some embodiments, a template other than the anti-sense RNA strand can serves as the ligation substrate, such that ligation and hybridization occur on a first template to form a combination oligomer, and the resulting combination oligomer is subsequently hybridized to the anti-sense RNA strand to form an anti-sense RNA complex.

In some embodiments, hybridization of contiguous oligomers can invoke the contiguous hybridization effect. The contiguous hybridization effect (see for example Stomakhin, V, et al., Nucleic Acids Research, 28(5):1193-8.) can occur when the thermal stability of the oligomers hybridized to contiguous regions results in thermal stability comparable to the hybridization of a full-length sequence comprising the aggregate of all the oligomers.

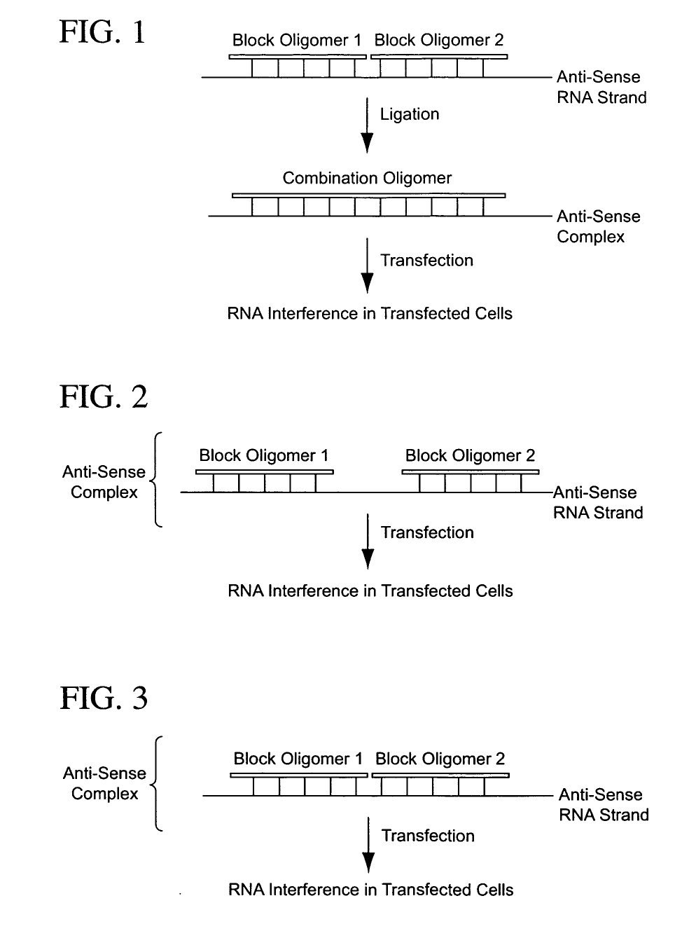

In some embodiments, the anti-sense complex will be generated by the template-based ligation of at least two oligomers, wherein the ligation of the two oligomers results in a combination oligomer (see for example FIG. 1).

In some embodiments, the anti-sense complex will be generated by hybridization of two adjacent but noncontiguous oligomers onto a template, wherein a gap exists between the at least two oligomers (see for example FIG. 2).

In some embodiments, the anti-sense complex can be generated by hybridization of two contiguous oligomers on the anti-sense RNA strand (see for example FIG. 3).

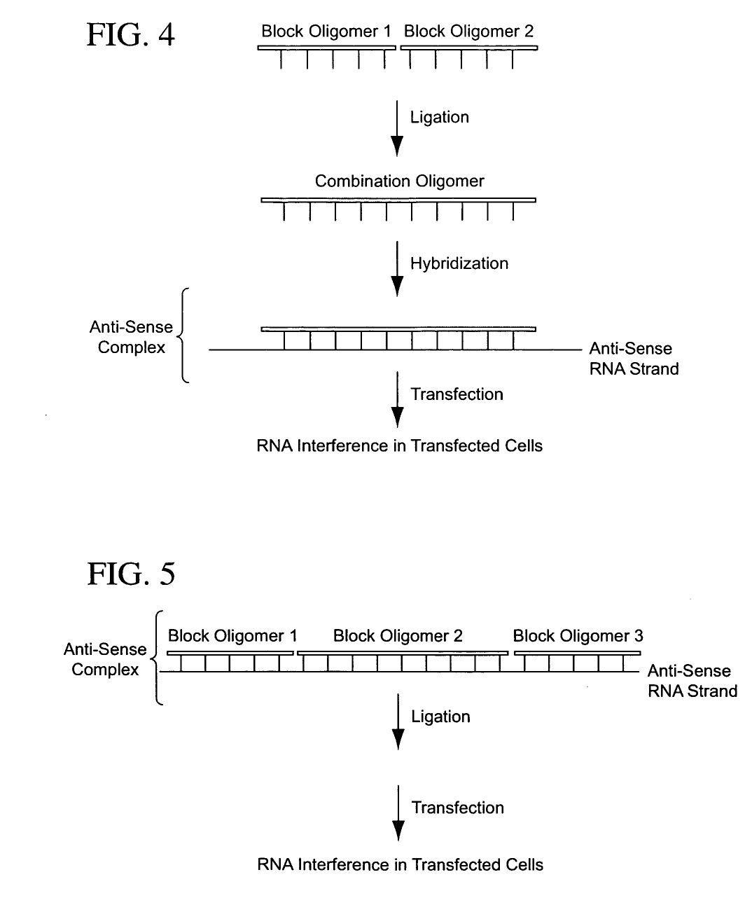

In some embodiments, a combination oligomer of the anti-sense complex can be generated by the non-templated ligation of at least two oligomers. Following ligation, the resulting combination oligomer can then hybridize to the anti-sense RNA strand, thereby resulting in an anti-sense complex (see for example FIG. 4).

In some embodiments, a combination oligomer is made when three block oligomers hybridize to adjacent and contiguous regions on a substrate and are ligated together (see for example FIG. 5).

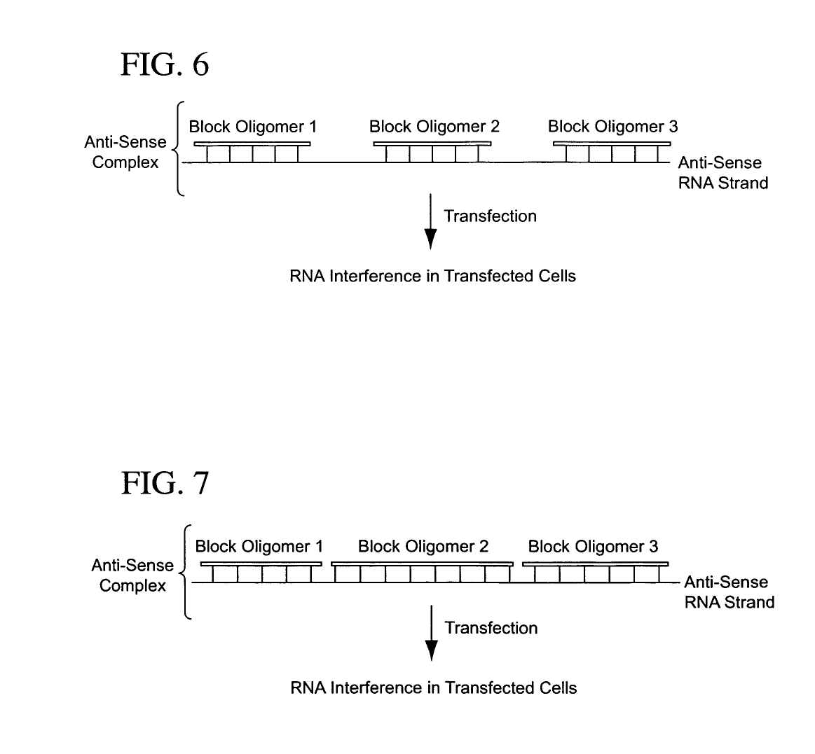

In some embodiments, three oligomers can be hybridized to adjacent, non-contiguous regions on a substrate to form an anti-sense complex (for example FIG. 6).

In some embodiments, three oligomers can hybridize to adjacent and contiguous regions on the anti-sense RNA strand to form an anti-sense complex in which the adjacent and contiguous molecules are not ligated together (see for example FIG. 7).

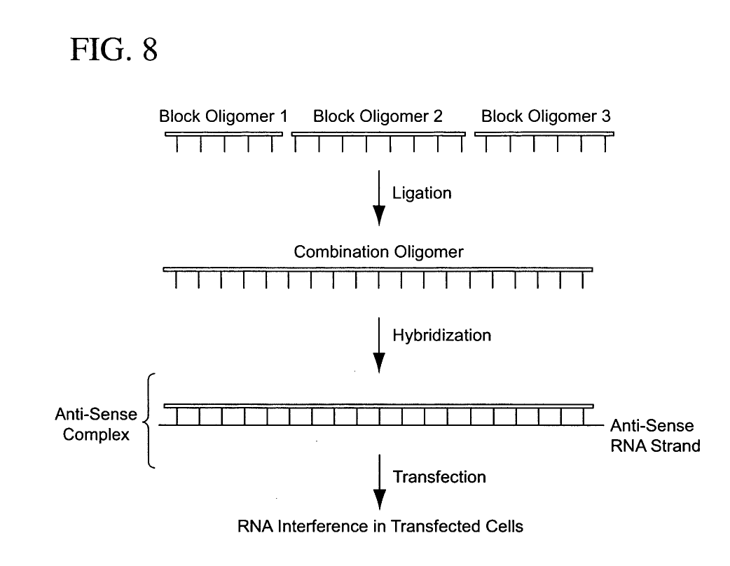

In some embodiments, three oligomers can be ligated together in a template-independent fashion to form a combination oligomer. The combination oligomer can be hybridized to the anti-sense RNA strand, thereby resulting in an anti-sense complex (see for example FIG. 8).

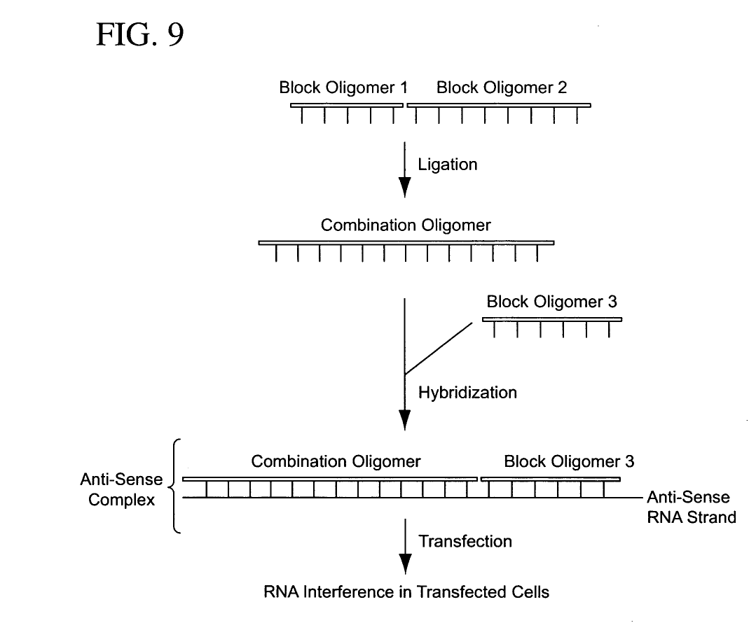

In some embodiments, three block oligomers can be employed, wherein two block oligomers are ligated together in a template independent fashion, and then are subsequently hybridized to an anti-sense RNA strand. Subsequently, a single oligomer can hybridize to a contiguous region on the same anti-sense RNA strand, and not be ligated (see for example FIG. 9). In the context of the present teachings, various permutations of the sort are contemplated for generating anti-sense complexes, wherein the relative timing, nature and presence of hybridization template, kind of ligation, the extent to which hybridized block oligomers have gaps or not, adjacently hybridized combination oligomers have gaps or not, adjacently hybridized, the presence of analogs, and the like, are varied in what will be appreciated as a large plurality of possibilities. Such maneuvers are clearly contemplated, and are routine techniques that would not require undue experimentation by one having ordinary skill in the art in light of the present teachings.

Composition of Oligomers

In some embodiments, a combination oligomer is made when a PNA block oligomer and at least one RNA block oligomer hybridize to adjacent and contiguous regions on a template. In some embodiments a hybridized PNA block oligomer is contiguously flanked by two RNA block oligomers, wherein the PNA block oligomer and the two RNA block oligomers can be ligated together, thereby forming a combination oligomer.

In some embodiments, two PNA block oligomers are hybridized to a template. In some embodiments, three PNA block oligomers are hybridized to a template. In some embodiments, more than three PNA block oligomers are hybridized to a template. In some embodiments, the PNA oligomers can be ligated together. In some embodiments, the PNA oligomers are not ligated together. In some embodiments, non-ligated PNA oligomers are contiguously hybridized to a template. In some embodiments, non-ligated PNA oligomers are hybridized non-contiguously to a template.

In some embodiments, the PNA block oligomers are chosen from libraries of PNA block oligomers comprising all possible 4⁴ tetramers. In some embodiments, the PNA block oligomers are chosen from libraries of PNA block oligomers comprising all possible 4⁵ pentamers. In some embodiments, the PNA block oligomers are chosen from libraries of PNA block oligomers comprising all possible 4⁶ hexamers. In some embodiments, the PNA block oligomers are chosen from libraries of PNA block oligomers comprising all possible 4⁷ heptamers. In some embodiments, the PNA block oligomers are chosen from libraries of PNA block oligomers comprising all possible 4⁸ octamers. In some embodiments, the PNA block oligomers are chosen from libraries of PNA block oligomers comprising all possible 4⁴ tetramers through 4⁸ octamers, or combinations thereof.

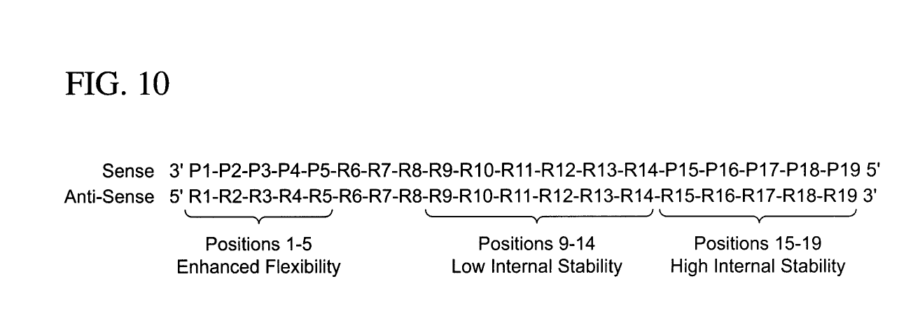

Some embodiments of the present teachings comprise a composition of matter comprising a sense strand combination oligomer as indicated in FIG. 10. Methods for the chemical assembly of PNAs are well known (see: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,063,569, Nielsen et al., Peptide Nucleic Acids; Protocols and Applications, Horizon Scientific Press, Norfolk England (1999) and PerSeptive Biosystems Product Literature. Methods for synthesizing PNA-DNA and PNA-RNA chimeras are well-known, and can be found for example in U.S. Pat. No. 6,063,569, U.S. Pat. No. 6,297,016, van der Laan et al., 1997, Tetrahedron Lettes, vol 38, no. 13, pp. 2249-2252, Greiner et al., Helvetica Chimica Acta, 2002, Vol. 85, pp. 2619-2626, Finn et al., Nucleic Acids Res. 1996 Sep. 1:24(17):3357-63, Verheijen et al., Nucleosides and Nucleotides, 18 (3), 493-508 (1999), Uhlmann, Biol. Chem., Vol. 379, pp. 1045-1052, Misra et al., Biochemistry (1998), 37, 1917-1925, Lutz et al., (1999) Nucleosides and Nucleotides 18(3), 393-401 (1999).

It will be appreciated that various configurations of oligomers can be employed in the context of the present teachings, in various contexts with generating the anti-sense complex as described supra, as well as in various contexts with the thermodynamic profiles of the anti-sense RNA strand as described infra.

Target Polynucleotide Sequence Selection

Some embodiments of the present teachings involve the selection of the region of the target polynucleotide sequence to which the anti-sense RNA strand of the anti-sense complex will hybridize. In general such selection methods comprise choosing a gene of interest, and using readily available software from the world wide web to determine all possible n-mer (for example, a 19-mer) antisense sequences without restriction to the known 2-nucleotide flanks (see for example, Jackson et al., 2003, Cell: 21:635-637, and Elbashir et al., 2002, Methods, 26:199-213), which can therefore provide the maximum number of possibilities. Conventional primer design software can then eliminate sequences with greater than or equal to n nucleotide complementarity (for example, 16 nucleotides, though any appropriate threshold can be employed) to mRNA sequences in the transcriptome of interest. Antisense sequences comprising a similarly high-degree of self-complementarity or those having intramolecular hairpin structures and homonucleotide runs of greater than equal to n nucleotides (for example, 4 G's in a row, though any appropriate threshold can be employed) can then be eliminated. The free-energies for positions about 9-14 of the remaining antisense sequences obtained thusly can then be computed, and then rank-ordering performed for those sequences in conformance with the thermodynamic profile of active siRNAs as described herein (also see Khvorova et al., 2003 for additional guidance, as well as co-filed U.S. Provisional Application Methods For The Computational Prediction of The Efficiency of Silencing RNA Constructs For RNA Interference to Ladunga et al.,). For the antisense sequence with the highest rank-order, the free energies for positions about 1-5, and for positions about 15-19, can then be computed with respect to hypothetical 5-mer hybrids of sense-PNA and anti-sense RNA. (The generation of melting curves for PNA-RNA hybrids could further inform these approaches, as described infra.) The thermodynamic profiles for positions about 1-5 and for positions about 15-19 are compared to the corresponding preferred kilocalorie/mole characteristics described infra. If conformance is judged to be inadequate, one or both profiles can be recalculated using the appropriate relatively stronger or weaker base-pairs in order to achieve adequate conformance. The PNA sequences representing the hypothetical positions about 1-5 and positions about 15-19 are then used as the 3′ and 5′ ends, respectively, of the chimeric PNA-RNA 19-mer to be synthesized. Synthesis of the chimeric PNA-RNA 19 mer thusly identified can be achieved by any appropriate methodology, as describe herein as well as by analogy with reported PNA-DNA chimeric synthesis (see for example Published P.C.T. Application WO96/40709).

It will be appreciated that these procedures for selection of the target polynucleotide sequence can be applied with readily achievable modification to any of the probe configurations described, for example, in FIGS. 1-10. Further, it will be appreciated that these procedures for selection of the target polynucleotide sequence can be applied in contexts where the anti-sense strand is a length other than 19 nucleotides, as described infra.

Thermodynamic Parameters of the Anti-Sense RNA Strand

As used herein, nucleobase positions are defined with respect to position 1 being the 5′ end of the anti-sense strand. It will further be appreciated that positions 1-5 of the 5′ end of the anti-sense strand, positions 9-14 of the 5′ end of the anti-sense strand, and positions 15-19 of the 5′ end of the anti-sense strand are approximations, and that one of skill in the art would be within the scope of the present teachings by shifting positions 1-5, for example, and its corresponding free energy values, to positions 2-6. Such a maneuver is clearly contemplated by the present teachings and falls within their scope.

In some embodiments the anti-sense RNA strand comprises 21 ribonucleotides. In some embodiments the anti-sense RNA strand comprises more than 21 ribonucleotides. In some embodiments the anti-sense RNA strand comprises fewer than 21 ribonucleotides. In some embodiments the anti-sense RNA strand comprises 21-30 ribonucleotides. In some embodiments the anti-sense RNA strand comprises 11-21 ribonucleotides. In some embodiments the anti-sense RNA strand comprises fewer than 11 ribonucleotides. In some embodiments the anti-RNA strand comprises greater than 30 ribonucleotides.

It will be appreciated that the regions comprising the enhanced flexibility profile, low internal stability profile, and high internal stability profile, will comprise thermodynamic values consistent with the nature of the moieties comprising the duplex. For example, RNA-RNA hybrids, all things being equal, are likely to have slightly different thermodynamic characteristics (e.g. melting curves) than a PNA-RNA hybrid. The values described herein assume an RNA-RNA hybrid (see for example Khvorova et al., 2003, Cell: 115:209-216). It will be appreciated that it is within the normal gambit of one having ordinary skill in the art to produce thermodynamic melting curves describing, for example, PNA-RNA hybrids, as well as other hybrids comprising for example universal nucleobases, and that such experimentation is routine and by no means undue. Calibrating the thermodynamic values described herein to such constructed melting curves is clearly within the scope of the present teachings.

It will be appreciated that the regions comprising the enhanced flexibility profile, low internal stability profile, and high internal stability profile, can vary depending on the length of the anti-sense RNA strand, wherein as a more general matter the 5′ end of the anti-sense RNA strand comprises a relatively enhanced flexibility profile, the middle regions of the anti-sense RNA strand comprises a relatively low internal stability profile, and the 3′ end of the anti-sense RNA strand comprises a relatively high internal stability profile.

In some embodiments of the present teachings, position about 1 to position about 8 of the 5′ end of the anti-sense strand comprise enhanced flexibility, with free energy values of about −6.0 kilocalories per mole to about −8.5 kilocalories per mole.

In some embodiments of the present teachings, position 1 to position 7 of the 5′ end of the anti-sense strand comprise enhanced flexibility, with free energy values of −6.0 to −8.5.

In some embodiments of the present teachings, position 1 to position 6 of the 5′ end of the anti-sense strand comprise enhanced flexibility, with free energy values of −6.0 to −8.5.

In some embodiments of the present teachings, position 1 to position 5 of the 5′ end of the anti-sense strand comprise enhanced flexibility, with free energy values of −6.0 to −8.5.

In some embodiments of the present teachings, position 1 to position 4 of the 5′ end of the anti-sense strand comprise enhanced flexibility, with free energy values of −6.0 to −8.5.

In some embodiments of the present teachings, position 1 to position 3 of the 5′ end of the anti-sense strand comprise enhanced flexibility, with free energy values of −6.0 to −8.5.

In some embodiments of the present teachings, position 2 to position 8 of the 5′ end of the anti-sense strand comprise enhanced flexibility, with free energy values of −6.0 to −8.5.

In some embodiments of the present teachings, position 3 to position 8 of the 5′ end of the anti-sense strand comprise enhanced flexibility, with free energy values of −6.0 to −8.5.

In some embodiments of the present teachings, position 4 to position 8 of the 5′ end of the anti-sense strand comprise enhanced flexibility, with free energy values of −6.0 to −8.5.

In some embodiments of the present teachings, position 5 to position 8 of the 5′ end of the anti-sense strand comprise enhanced flexibility, with free energy values of −6.0 to −8.5.

In some embodiments of the present teachings, position 6 to position 8 of the 5′ end of the anti-sense strand comprise enhanced flexibility, with free energy values of −6.0 to −8.5.

In some embodiments of the present teachings, position 1 to position 8 of the 5′ end of the anti-sense strand comprise enhanced flexibility, with free energy values of −6.0 to −8.5.

In some embodiments of the present teachings, position 1 to position 8 of the 5′ end of the anti-sense strand comprise enhanced flexibility, with free energy values of −6.5 to −8.5.

In some embodiments of the present teachings, position 1 to position 8 of the 5′ end of the anti-sense strand comprise enhanced flexibility, with free energy values of −7.0 to −8.5.

In some embodiments of the present teachings, position 1 to position 8 of the 5′ end of the anti-sense strand comprise enhanced flexibility, with free energy values of −7.5 to −8.5.

In some embodiments of the present teachings, position 1 to position 8 of the 5′ end of the anti-sense strand comprise enhanced flexibility, with free energy values of −8.0 to −8.5.

In some embodiments of the present teachings, position 1 to position 8 of the 5′ end of the anti-sense strand comprise enhanced flexibility, with free energy values of −6.0 to −8.0.

In some embodiments of the present teachings, position 1 to position 8 of the 5′ end of the anti-sense strand comprise enhanced flexibility, with free energy values of −6.0 to −7.5.

In some embodiments of the present teachings, position 1 to position 8 of the 5′ end of the anti-sense strand comprise enhanced flexibility, with free energy values of −6.0 to −7.0.

In some embodiments of the present teachings, positions 1 to position 8 of the 5′ end of the anti-sense strand comprise enhanced flexibility, with free energy values of −6.0 to −6.5.

In some embodiments of the present teachings, position about 9 to position about 14 of the 5′ end of the anti-sense strand further comprise a low internal stability profile, with free energy values of about −8.0 to about −9.0 kilocalories per mole.

In some embodiments of the present teachings, positions 9-14 of the 5′ end of the anti-sense strand further comprise a low internal stability profile, with free energy values of −8.0 to −8.5.

In some embodiments of the present teachings, positions 9-14 of the 5′ end of the anti-sense strand further comprise a low internal stability profile, with free energy values of −8.5 to −9.0.

In some embodiments of the present teachings, positions 10-14 of the 5′ end of the anti-sense strand further comprise a low internal stability profile, with free energy values of −8.0 to −9.0.

In some embodiments of the present teachings, positions 10-14 of the 5′ end of the anti-sense strand further comprise a low internal stability profile, with free energy values of −8.0 to −8.5.

In some embodiments of the present teachings, positions 10-14 of the 5′ end of the anti-sense strand further comprise a low internal stability profile, with free energy values of −8.5 to −9.0.

In some embodiments of the present teachings, positions 11-14 of the 5′ end of the anti-sense strand further comprise a low internal stability profile, with free energy values of −8.0 to −9.0.

In some embodiments of the present teachings, positions 11-14 of the 5′ end of the anti-sense strand further comprise a low internal stability profile, with free energy values of −8.0 to −8.5.

In some embodiments of the present teachings, positions 11-14 of the 5′ end of the anti-sense strand further comprise a low internal stability profile, with free energy values of −8.5 to −9.0.

In some embodiments of the present teachings, positions 12-14 of the 5′ end of the anti-sense strand further comprise a low internal stability profile, with free energy values of −8.0 to −9.0.

In some embodiments of the present teachings, positions 12-14 of the 5′ end of the anti-sense strand further comprise a low internal stability profile, with free energy values of −8.0 to −8.5.

In some embodiments of the present teachings, positions 12-14 of the 5′ end of the anti-sense strand further comprise a low internal stability profile, with free energy values of −8.5 to −9.0.

In some embodiments of the present teachings, positions 9-13 of the 5′ end of the anti-sense strand further comprise a low internal stability profile, with free energy values of −8.0 to −9.0.

In some embodiments of the present teachings, positions 9-13 of the 5′ end of the anti-sense strand further comprise a low internal stability profile, with free energy values of −8.0 to −8.5.

In some embodiments of the present teachings, positions 9-13 of the 5′ end of the anti-sense strand further comprise a low internal stability profile, with free energy values of −8.5 to −9.0.

In some embodiments of the present teachings, positions 9-12 of the 5′ end of the anti-sense strand further comprise a low internal stability profile, with free energy values of −8.0 to −9.0.

In some embodiments of the present teachings, positions 9-12 of the 5′ end of the anti-sense strand further comprise a low internal stability profile, with free energy values of −8.5 to −9.0.

In some embodiments of the present teachings, position about 15 to position about 19 of the 5′ end of the anti-sense strand further comprises a high internal stability profile, with free energy values from about −8.5 to about −9.5 kilocalories per mole.

In some embodiments of the present teachings, positions 15-19 of the 5′ end of the anti-sense strand further comprises a high internal stability profile, with free energy values from about −8.5 to about −9.0.

In some embodiments of the present teachings, positions 15-19 of the 5′ end of the anti-sense strand further comprises a high internal stability profile, with free energy values from about −9.0 to about −9.5.

In some embodiments of the present teachings, positions 16-19 of the 5′ end of the anti-sense strand further comprises a high internal stability profile, with free energy values from about −8.5 to about −9.5.

In some embodiments of the present teachings, positions 16-19 of the 5′ end of the anti-sense strand further comprises a high internal stability profile, with free energy values from about −8.5 to about −9.0.

In some embodiments of the present teachings, positions 16-19 of the 5′ end of the anti-sense strand further comprises a high internal stability profile, with free energy values from about −9.0 to about −9.5.

In some embodiments of the present teachings, positions 17-19 of the 5′ end of the anti-sense strand further comprises a high internal stability profile, with free energy values from about −8.5 to about −9.0.

In some embodiments of the present teachings, positions 17-19 of the 5′ end of the anti-sense strand further comprises a high internal stability profile, with free energy values from about −9.0 to about −9.5.

In some embodiments of the present teachings, positions 15-18 of the 5′ end of the anti-sense strand further comprises a high internal stability profile, with free energy values from about −8.5 to about −9.5.

In some embodiments of the present teachings, positions 15-18 of the 5′ end of the anti-sense strand further comprises a high internal stability profile, with free energy values from about −8.5 to about −9.0.

In some embodiments of the present teachings, positions 15-18 of the 5′ end of the anti-sense strand further comprises a high internal stability profile, with free energy values from about −9.0 to about −9.5.

In some embodiments of the present teachings, positions 15-17 of the 5′ end of the anti-sense strand further comprises a high internal stability profile, with free energy values from about −8.5 to about −9.5.

In some embodiments of the present teachings, positions 15-17 of the 5′ end of the anti-sense strand further comprises a high internal stability profile, with free energy values from about −8.5 to about −9.0.

In some embodiments of the present teachings, positions 15-17 of the 5′ end of the anti-sense strand further comprises a high internal stability profile, with free energy values from about −9.0 to about −9.5.

Kits

Some embodiments of the present teachings comprise kits for performing the methods of the present teachings utilizing the compositions of the present teachings. In some embodiments, such kits can comprise the anti-sense complex, along with, optionally, transfection reagents for introducing the composition into cells of interest. In some embodiments, the kits of the present teachings comprise block oligomers and/or anti-sense RNA, along with the appropriate buffers, and ligation reagents for producing an anti-sense complex. In some embodiments, the kits of the present teachings comprise an anti-sense complex and a delivery means for introducing the anti-sense complex into cells. In some embodiments, the kits of the present teachings comprise target cells altered in such ways that are known in the art of increase the uptake of exogenous molecules (e.g. competent cells). In some embodiments, the kits can further comprise reagents for assessing reduction of the target polynucleotide sequence, for example, Taq-Man real-time PCR reagents commercially available from Applied Biosystems.

### 4. EXAMPLES

### Example 1

mRNA encoded by the human MDR1 gene (accession no. NM_{—}000927) is analyzed for possible target regions using any of a number of available algorithms (see for example, siDESIGN Center at world wide web site dharmacon.com). The user-selected parameters for this algorithm are specified as amino acid start sites that target the open reading frame, have 4-of-19 through 10-of-19 G/C basepairs, and, allow 3 or more consecutive A,G,C, or T (U) bases. From the approximately 125 sequences thusly found, a total of about 30 target regions remain after automated BLAST elimination. Inspection of these approximately 30 sequences leads to selection of cases wherein, on a qualitative basis, there is relatively high A/T content in about 8 basepairs at the 5′ end relative to the 3′ end. This preliminary visual filter is used to select for candidate target regions that are more likely to fulfill the thermodynamic profile described in the present teachings. The 4 target regions thus selected for further analysis are shown in Table 1 in the 5′ to 3′ direction and are numbered SEQ NO: 1-4 as the open reading frame position of the 5′ base. The thermodynamic (free-energy) values for basepairs 1-19 of the corresponding anti-sense RNA were calculated as described in the literature (Khvorova et al., 2003) and are listed in Table 1 as kcal/mol. On the basis of these free-energy values, the anti-sense RNA for SEQ ID:1 is considered to have adequate enhanced flexibility at the 5′ end but fails to meet the criterion of high internal stability at the 3′ end. By contrast, the anti-sense RNAs for SEQ ID. NO:2 and SEQ ID NO:3 both fail to meet the criterion for enhanced flexibility at the 5′ end yet have the required high internal-stability at the 3′ end. Of note, the anti-sense RNA for SEQ ID NO:4 is found to meet the criteria for enhanced flexibility at the 5′ end, as well as high internal-stability of the 3′ end. SEQ ID NO:4 is therefore chosen for synthesis as an antisense-RNA sense-PNA hybrid and evaluation for RNAi activity in vitro as described infra.

From a library of 1,024 possible 5-mer PNA sequences, the three 5-mers shown below are selected such that each is complementary to the three segments of the antisense-RNA 19-mer. In this illustrative construction, there are two 1-base gaps between the PNAs and a 1-base RNA overhang at the 3′ end. Other compositions can be made with different gap sizes or locations, or overhands, or with contiguous basepairing, which includes use of other n-mer PNA libraries, as described supra.
Based on SEQ ID NO:4 Sense PNA (5-mers) taatt tatcc catct SEQ ID NO:5 Antisense RNA (19-mer) 5′auuaau----auagga----guagaua3′

The PNA and RNA components are hybridized in appropriate molar ratios at a hybrid concentration of 1 uM in water by first denaturing for 2 minutes at about 90C and then slow cooling to room temperature or below. Of note, the use of water, as opposed to a physiological (or other) salt solution, in combination with the relatively low concentration of PNA-RNA hybrid is intended to favor PNA-RNA hybridization relative to potentially problematic RNA-RNA hybridization. Commercially available Lipofectamine (Invitrogen, or an analogous cationic lipid, or a cell-type specific cationic lipid) is added to the PNA-RNA hybrid to form transfectable complexes using general procedures and methods for optimization describe in the literature (Braasch and Corey, 2001, Cell, 115:209-216). Aliquots of each transfectable complex are added to MDR1-expressing cells such that the final concentration of PNA-RNA hybrid covers a range of about 0, 5, 10, 20, 40, and 80 nM. Control samples include but are not limited to a PNA-RNA hybrid having no significant antisense-RNA complementarity to the human transcriptome, unhybridized PNAs and RNAs, and Lipfectamine alone. After incubation for 0, 24, 48, 72, and 96 hours, aliquots of the cell culture are removed for quantitative analysis of MDR1 expression by use of real-time fluorogenic assays such as TaqMan™ (Applied Biosystems) and protein analysis by Western blots or mass spectrometric methods such as ICAT™ (Applied Biosystems).

### Example 2

The functional significance of a SNP in the MDR1 gene can be studied in vitro, or abrogated in clinical applications, by use of antisense-RNA sense-PNA hybrids as described in Example 1, but with the following modification due to restricted mRNA targeting associated with a given SNP locus in the MDR1 gene. The G2677T/A locus encodes mRNA having either Ala or Ser/Thr at amino acid position 893. In the mRNA sequence given in the NCBI/NIH database for MDR1 (accession no. NM_{—}000927) shown below, it can be seen that the mRNA codes for the Ser (S) variant at position 893 (bold underlined). Since each amino acid is encoded by a triplet RNA codon, the RNA sequence corresponding to amino acid position 893 and 6 amino acids on either side, or 18 ribonucleotide flanking residues (lower case). This indicates a total of 39 (18+3+18) ribonucleotide residues in the mRNA locus from which to select target region sequences.
SEQ ID NO:6 KMLSGQALKDkkelegSgkiateAlENFRTWSLTQ

The mRNA corresponding to this 39 ribonucleotide region is analyzed for the target region which affords an anti-sense RNA having a desireable thermodynamic profile. In the absence of actual thermodynamic values for inosine, which serves as a universal base (see below), the thermodynamic calculation uses a phantom mock value of −2.0 kcal/mol for the inosine-containing base-pair, regardless of the neighboring basepair. While this mock value might produce slightly inaccurate absolute values, the thermodynamic profiles are qualitatively compared with the enhanced flexibility and internal-stability criteria described considered in Example 1. The antisense-RNA thus chosen is synthesized with inosine in the appropriate codon position relative to Ala893Ser/Thr (see Ohtsuka et al., 1985, J.B.C. 260: 2605-2608 for further teachings on the application of universal bases). The subsequent line of experimentation is essentially similar to that described in Example 1.

TABLE 1
Selected MDR1 mRNA Sequences and Calculated Thermodynamic Values
(kcal/mol) of Corresponding siRNA Basepairs 1-19 (nd = not determined).

| | Seq. No. | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| SEQ ID | a | u | u | u | a | a | c | u | g | a | a | u |
| NO: 1 | | | | | | | | | | | | |
| kcal/mol | 5.8 | 7.1 | 7.9 | 9 | 10 | 10 | 9 | 9.3 | 10 | 12 | 14 | 15 |
| SEQ ID | a | u | a | a | a | g | c | g | a | c | u | g |
| NO: 2 | | | | | | | | | | | | |
| kcal/mol | 6.6 | 9.3 | 10 | 12 | 13 | 13 | 11 | 12 | 10 | 9 | 9.3 | 9.5 |
| SEQ ID | u | g | u | u | u | c | g | c | u | a | u | u |
| NO: 3 | | | | | | | | | | | | |
| kcal/mol | 9.4 | 11 | 12 | nd | nd | nd | nd | nd | nd | nd | nd | nd |
| SEQ ID | u | a | a | u | u | a | u | a | u | c | c | u |
| NO: 4 | | | | | | | | | | | | |
| kcal/mol | 6.1 | 5.8 | 6.1 | 6.1 | 7.6 | 9.5 | 10 | 10 | 12 | 11 | 8.9 | 9.6 |

| | Seq. No. | | | | | | | | | | |
|---|---|---|---|---|---|---|---|---|---|---|---|
| | 13 | 14 | 15 | 16 | 17 | 18 | 19 | 20 | 21 | 22 | 23 |
| SEQ ID | g | g | c | c | u | u | a | u | u | u | u |
| NO: 1 | | | | | | | | | | | |
| kcal/mol | 13 | 12 | 8.8 | 6.6 | 5.8 | 5.8 | 4.4 | | | | |
| SEQ ID | a | a | u | g | u | u | c | u | c | a | c |
| NO: 2 | | | | | | | | | | | |
| kcal/mol | 7.9 | 9.4 | 11 | 10 | 10 | 11 | 11 | | | | |
| SEQ ID | c | a | a | a | u | u | g | g | c | u | u |
| NO: 3 | | | | | | | | | | | |
| kcal/mol | nd | nd | 8.8 | 12 | 12 | 12 | 12 | | | | |
| SEQ ID | u | c | a | u | c | u | a | u | g | g | u |
| NO: 4 | | | | | | | | | | | |
| kcal/mol | 10 | 9.2 | 8.1 | 7 | 9.9 | 10 | 10 | | | | |

While the present teachings have been described in terms of these exemplary embodiments, examples, and figures, the skilled artisan will readily understand that numerous variations and modifications of these exemplary embodiments are possible without undue experimentation. All such variations and modifications are within the scope of the current teachings.

What is claimed is:

1. A method of reducing the amount of a target polynucleotide sequence comprising,
providing,
a. an anti-sense strand, wherein the anti-sense strand is complementary to a target region, and,
b. at least two block oligomers, wherein the at least two block oligomers further comprise PNA, wherein the block oligomer can hybridize to the anti-sense strand to form an anti-sense complex;
delivering the anti-sense complex to a sample comprising the target polynucleotide sequence, and;
reducing the amount of the target polynucleotide sequence.

2. The method according to claim 1 wherein the least two block oligomers comprise pentameric PNAs.

3. The method according to claim 1 wherein the at least two block oligomers comprise tetrameric PNAs.

4. The method according to claim 1 wherein at least one of the at least two block oligomers comprises a pentameric PNA and at least one of the at least two block oligomers comprises a tetrameric PNA.

5. The method according to claim 1 wherein the at least two block oligomers are hybridized to adjacent and non-contiguous regions on the anti-sense strand.

6. The method according to claim 1 wherein the at least two block oligomers are hybridized to adjacent and contiguous regions on the at least one anti-sense strand.

7. The method according to claim 1 wherein the at least two block oligomers are ligated together, thereby forming a combination oligomer, wherein the combination oligomer is hybridized on the anti-sense strand.

8. A method of reducing the amount of at least one target polynucleotide sequence comprising,
providing,
i. an anti-sense strand, wherein the anti-sense strand is complementary to a target region, and,
ii. a combination oligomer, wherein the combination oligomer further comprises a PNA nucleobase sequence, a first RNA nucleobase sequence, and a second RNA nucleobase sequence, wherein the combination oligomer hybridizes to the anti-sense strand, to form an anti-sense complex,
delivering the anti-sense complex to a sample comprising the target polynucleotide sequence, and;
reducing the amount of the target polynucleotide sequence.

9. The method according to claim 8 wherein the first RNA nucleobase sequence and the second RNA nucleobase sequence flank the PNA nucleobase sequence.

10. The method according to claim 9 wherein the PNA nucleobase sequence further comprises about 9 PNA nucleobases.

11. The method according to claim 9 wherein the first RNA nucleobase sequence comprises about 5 RNA nucleobases,

12. The method according to claim 9 wherein the second RNA nucleobase sequence comprises about 5 RNA nucleobases.

13. The method according to claim 9 wherein position about 1 to position about 5 of the anti-sense strand comprises enhanced flexibility.

14. The method according to claim 13 wherein the enhanced flexibility comprises a free energy value of about −6.5 to about −8.0 kilocalories per mole.

15. The method according to claim 9 wherein position about 9 to position about 14 of the anti-sense strand comprise a low internal stability profile.

16. The method according to claim 15 wherein the low internal stability profile comprises a free energy value of about −8.0 to about −9.0 kilocalories per mole.

17. The method according to claim 9 wherein position about 15 to position about 19 of the anti-sense strand comprise a high internal stability profile.

18. The method according to claim 17 wherein the high internal stability profile comprises a free energy value of about −8.5 to about −9.5 kilocalories per mole.

19. A composition of matter comprising,
a. an anti-sense strand complementary to a target region, and,
b. a first block oligomer, a second block oligomer, and a third block oligomer, wherein the first block oligomer and the third block oligomer each comprise an RNA nucleobase sequence, and the second block oligomer comprises a PNA nucleobase sequence, wherein the first block oligomer, the second block oligomer, and the third block oligomer are hybridized to the anti-sense strand.

20. The composition according to claim 19 wherein the first block oligomer, the second block oligomer, and the third block oligomer are ligated together to form a combination oligomer.

21. The composition according to claim 19 wherein the second block oligomer is flanked by the first block oligomer and the third block oligomer.

22. The composition according to claim 19 wherein the second block oligomer comprises PNA.

23. The composition according to claim 22 wherein the second block oligomer further comprises about 9 PNA nucleobases.

24. The composition according to claim 19 wherein the first block oligomer comprises RNA.

25. The composition according to claim 24 wherein the first block oligomer comprises about 5 RNA nucleobases.

26. The composition according to claim 19 wherein the third block oligomer comprises RNA.

27. The composition according to claim 26 wherein the first block oligomer comprises about 5 RNA nucleobases.

28. The composition according to claim 19 wherein the PNA nucleobase sequence of the second block oligomer further comprises about 9 PNA nucleobases, wherein the RNA nucleobase sequence of the first block oligomer comprises about 5 RNA nucleobases, wherein the RNA nucleobase sequence of the third block oligomer comprises about 5 RNA nucleobases, and wherein the first block oligomer and the third block oligomer flank the second block oligomer.

29. The composition according to claim 19 wherein position about 1 to position about 5 of the anti-sense strand comprises enhanced flexibility.

30. The composition according to claim 29 wherein the enhanced flexibility comprises a free energy value of about −6.5 to about −8.0 kilocalories per mole.

31. The composition according to claim 19 wherein position about 9 to position about 14 of the anti-sense strand further comprises a low internal stability profile.

32. The composition according to claim 31 wherein the low internal stability profile comprises a free energy value of about −8.0 to about −9.0 kilocalories per mole.

33. The composition according to claim 19 wherein position about 15 to position about 19 of the anti-sense strand further comprises a high internal stability profile.

34. The composition according to claim 33 wherein the high internal stability profile comprises a free energy value of about −8.5 to about −9.5 kilocalories per mole.

35. A kit for reducing the amount of a target polynucleotide sequence comprising,
a means for transfecting, and,
an anti-sense complex.

36. The kit according to claim 35 comprising competent cells.

37. A kit for reducing the amount of a target polynucleotide sequence comprising,
an anti-sense complex, and,
a transfection agent.

38. The kit according to claim 37 wherein the anti-sense complex comprises an anti-sense strand, and a combination oligomer, wherein the combination oligomer comprises a PNA block oligomer flanked by a first RNA block oligomer and a second RNA block oligomer.

39. The kit according to claim 37 wherein the anti-sense complex comprises an anti-sense strand, and at least two block oligomers, wherein the at least two block oligomers are PNA block oligomers.

40. The kit according to claim 37 further comprising competent cells.


SEQUENCE LISTING (6 sequences)

SEQ ID NO: 1 - RNA, 23 nt, Human
auuuaacugaauggccuuauuuu

SEQ ID NO: 2 - RNA, 23 nt, Human
auaaagcgacugaauguucucac

SEQ ID NO: 3 - RNA, 23 nt, Human
uguuucgcuauucaaauuggcuu

SEQ ID NO: 4 - RNA, 23 nt, Human
uaauuauauccuucaucuauggu

SEQ ID NO: 5 - RNA, 19 nt, Human
auuaauauaggaguagaua

SEQ ID NO: 6 - protein, 36 aa, Human
KMLSGQALKDKKELEGSGKIATEAIENFRTVVSLTQ